(19) United States
(12) Patent Application Publication
(10) Pub. No.: US 2023/0062413 A1
(43) Pub. Date: Mar. 2, 2023

(54) RIGID FRAME BAGS

(71) Applicant: ENTEGRIS, INC., Billerica, MA (US)

(72) Inventor: Tate Vangsgard, Oakdale, MN (US)

(21) Appl. No.: 17/893,658

(22) Filed: Aug. 23, 2022

Related U.S. Application Data
(60) Provisional application No. 63/236,994, filed on Aug. 25, 2021.

(51) Int. Cl. B01L 3/00

(52) U.S. Cl. CPC B01L 3/505; B01L 2200/026; B01L 2300/042; B01L 2300/16

(57) ABSTRACT

A bioprocess bag for containing bioprocess fluids includes a frame having a first opening and a second opening, a first layer of film bonded to the frame to cover the first opening, and a second layer of film bonded to the frame to cover the second opening. The frame, the first layer of film, and the second layer of film form an enclosed space for containing the bioprocess fluids. The frame is disposed between the first layer of film and the second layer of film.

### PRIORITY

This document claims priority to U.S. Provisional Application No. 63/236,994 with a filing date of Aug. 25, 2021.

### FIELD

This disclosure is directed to bioprocess bags and/or to bags for containing pharmaceutical or biological fluids and the like; particularly to bags with a rigid frame providing support to the bag during freezing, thawing, and/or frozen shipping processes.

### BACKGROUND

Chemical and/or biological processes can be carried out, or their results contained in bags such as bags containing pharmaceutical or biological fluids, bioprocess bags, and the like. Process materials or materials resulting from the process may need to be frozen or otherwise kept at low temperature. Bags may need to be supported through cooling or freezing processes to ensure the integrity of the bag or maintenance of a desirable shape.

### SUMMARY

This disclosure is directed to bioprocess bags and/or to bags for containing pharmaceutical or biological fluids and the like; particularly to bags with a rigid frame providing support to the bag during freezing, thawing, and/or frozen shipping processes.

A particular type of bag (e.g., made of fluoropolymer or the like) has been used for plate freezing and thawing applications, with bag holders being designed and built around the need of the fluoropolymer bags. As the bag continues to shrink in volume for use in some small volume applications, eventually a ratio between how large the fitting is and how small the bag is can become undesirable.

Embodiments disclosed herein can provide a bag with a rigid frame (or shell), reducing the overall cost since there is no need for a separate bag holder, and can achieve a constant height of fluid/bag (e.g., a consistent freeze profile throughout the volume), providing a stackable and compact structure that can increase the usability of the bag, achieve efficient freeze and/or thaw throughput, and ensure ease of handling and transport and increased gas transmission.

In an embodiment, a bioprocess bag for containing bioprocess fluids includes a frame having a first opening and a second opening, a first layer of film bonded to the frame to cover the first opening, and a second layer of film bonded to the frame to cover the second opening. The frame, the first layer of film, and the second layer of film form an enclosed space for containing the bioprocess fluids. The frame is disposed between the first layer of film and the second layer of film.

In specific embodiments, the frame, the first layer of film, and the second layer of film are made of the same material or compatible materials. For example, the frame, the first layer of film, and the second layer of film can each be made of a fluoropolymer. In some embodiments, the first layer of film and/or the second layer of film can be a single layer film or a multiple-layer film.

Furthermore, the minimum and maximum thickness of the frame can vary depending, for example, on the desired final volume. For example, the minimum thickness of the frame can be at or about 30 thousandth of an inch and the maximum thickness of the frame can be, for example, at or about three inches.

The minimum and maximum thickness of the first layer of film or the second layer of film can also vary depending, for example, on the properties of the film and/or the conditions to which the films will be exposed. For example, the minimum thickness of the first layer, the second layer, or both can be at or about three thousandth of an inch. The maximum thickness of the first layer of film and/or the second layer of film is at or about 25 thousandth of an inch.

In some embodiments, the bag further includes at least one fitting. The at least one fitting is configured to fill the bag with the bioprocess fluids and/or drain the bioprocess fluids from the bag. In an embodiment, the at least one fitting and the frame are integrally formed using, for example, injection molding.

The bag may further include a cap, which can have at least one fitting cover configured to protect the at least one fitting. The cap can be configured to cover the first layer of film that covers the first opening or the second layer of film that covers the second opening of the frame. In some embodiments, the cap can be made of plastic or stainless steel and preferably has a flat surface.

In an embodiment, the frame includes an interlocking mechanism. For example, the bag may further include a cap having an interlocking mechanism. The interlocking mechanism of the frame and the interlocking mechanism of the cover can be configured to interlock with each other using a snap-fit connection.

In another embodiment, the bioprocess bag contains bioprocess fluids and includes a frame having a front opening and a plurality of additional openings, a plate configured to cover the front opening, and a first layer of film, a second layer of film, and a third layer of film bonded to the frame to cover each of the additional openings of the plurality of additional openings, respectively. For this embodiment, the frame, the plate, the first layer of film, the second layer of film, and the third layer of film form an enclosed space for containing the bioprocess fluids. Alternatively, the bag can further include a fourth layer of film and a fifth layer of film. The first layer of film, the second layer of film, the third layer of film, the fourth layer of film, and the fifth layer of film are bonded to the frame to cover each opening of the plurality of openings, respectively. The frame, the plate, the first layer of film, the second layer of film, the third layer of film, the fourth layer of film, and the fifth layer of film form the enclosed space for containing the bioprocess fluids. For this embodiment, the plate can include at least one fitting, which can be configured to fill the bag with the bioprocess fluids and/or drain the bioprocess fluids from the bag. Also, the plate and the frame can be integrally formed, such as using injection molding.

This disclosure is also directed to a method of forming a bioprocess bag for containing bioprocess fluids. The method includes forming a frame having a first opening and a second opening, forming a first layer of film and a second layer of film, bonding the first layer of film to the frame to cover the first opening, bonding the second layer of film to the frame to cover the second opening, and forming an enclosed space for containing the bioprocess fluids by the frame, the first layer of film, and the second layer of film. The frame is disposed between the first layer of film and the second layer of film.

In an embodiment, the method further includes forming a cap having a flat surface and interlocking the cap and the frame using a snap-fit connection. The method may further include covering at least one fitting with at least one fitting cover of the cap. The method may further include covering the first layer of film that covers the first opening or the second layer of film that covers the second opening of the frame with the cap.

### DRAWINGS

References are made to the accompanying drawings that form a part of this disclosure and which illustrate various embodiments in which the bioprocess bags and methods described in this specification can be practiced. It should be understood that these drawing are not necessarily drawn to scale and may, in some cases, present a somewhat simplified representation of various preferred features illustrative of the basic principle of the disclosure. The specific design features of the present disclosure, including, for example, specific dimensions, orientations, locations, and shapes, will be determined in part by the intended application and use environment. Non-limiting and non-exhaustive descriptions are described with reference to the following drawings.

FIG. 1 is a top perspective view of a rigid frame bag, according to an embodiment.

FIG. 2 is a bottom perspective view of the rigid frame bag of FIG. 1, according to an embodiment.

FIG. 3 is a top perspective view of a rigid frame bag, according to another embodiment.

FIG. 4 is a top perspective view of a rigid frame bag, according to yet another embodiment.

FIG. 5 is a top perspective view of a rigid frame bag, according to yet another embodiment.

FIG. 6 is a top perspective view of a rigid frame bag, according to yet another embodiment.

FIG. 7 is a top perspective view of a rigid frame bag, according to yet another embodiment.

FIG. 8 is a perspective view of rigid frame bags stacked together, according to an embodiment.

FIG. 9 is a perspective view of rigid frame bags stacked together, according to another embodiment.

FIG. 10 is a top perspective view of a rigid frame bag, according to an embodiment.

FIG. 11 is a top perspective view of a cassette, according to an embodiment.

FIG. 12 is a bottom perspective view of the cassette of FIG. 11, according to an embodiment.

FIG. 13 is a perspective view of a 3D rigid frame bag, according to an embodiment.

FIG. 14 is a perspective view of a 3D rigid frame bag, according to another embodiment.

FIG. 15 is a flowchart illustrating a method of forming a bioprocess bag for containing bioprocess fluids, according to an embodiment.

Like reference numbers represent like parts throughout.

### DETAILED DESCRIPTION

This disclosure is directed to bioprocess bags and/or to bags for containing pharmaceutical or biological fluids and the like; particularly to bags with a rigid frame providing support to the bag during freezing, thawing, and/or frozen shipping processes.

The following definitions are applicable throughout this disclosure. As defined herein, the phrase “frame” may refer to e.g., a rigid structure that at least partially surrounds or encloses an object or a space within an object. In an embodiment, a frame can be thick enough to be injection molded and provide rigidity to the bioprocess bag. As an example, the frame can have a minimum thickness of at or about 0.03 inches. Also, the frame can have a maximum thickness of at or about 3 inches. In an embodiment, the frame can have a width or diameter (if circular) of at or about 1.5 inches.

As defined herein, the term “film” can refer to e.g., a thin flexible piece of plastic or any other suitable material. Preferably, the film is thin enough to be flexible to allow the fluid contained in a bag to expand and/or contract as needed, such as during filling or dispensing, yet is thick enough to withstand various handling and processing conditions without breaking easily. The film typically goes through a manufacturing process that is different from injection molding. In an embodiment, the film has a minimum thickness of at or about 0.003 inches. In another embodiment, the film has a maximum thickness of at or about 0.025 inches.

Some embodiments of the present disclosure are described in detail with reference to the accompanying drawings so that the advantages and features of the present application can be more readily understood by those skilled in the art. The terms “near”, “far”, “top”, “bottom”, “left”, “right” and the like described in the present application are defined according to the typical observation angle of a person skilled in the art and for the convenience of the description. These terms are not limited to specific directions.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

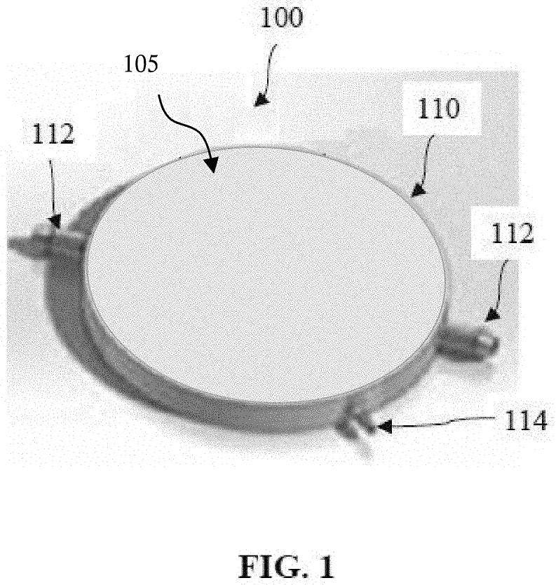

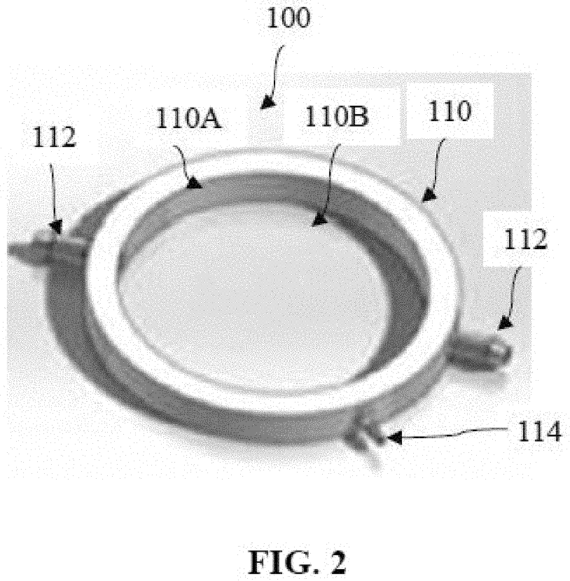

FIGS. 1-7 and 10 are perspective views of various frames and rigid frame bags, according to some embodiments. For example, FIG. 1 is a top perspective view of rigid frame bag 100, and FIG. 2 is a bottom perspective view of the rigid frame bag of FIG. 1, without a film layer to show the features of the frame.

Thus, as shown in FIG. 1, rigid frame bag 100 includes frame 110 and layer(s) of film(s) 105 (only the top layer is shown) bonded to the frame 110 to form the rigid frame bag 100. In this embodiment, the frame and the film(s) can be made of the same material or compatible materials. It will be appreciated that compatible materials can be referred to as the same material or as different materials that have similar enough properties for the materials to be bonded (or welded, adhered, or the like) to each other. For example, frame 110 and/or film 105 can be made of fluoropolymer, which is an organic polymer containing fluorine atoms such as polytetrafluoroethylene. It will be appreciated that the fluoropolymer may be rigid or stiff at room temperature, but it does not get stiffer when it is under freezing temperature. As such, the fluoropolymer bag can therefore be used for freezing and thawing applications. It will also be appreciated that rigid frame bag 100 can be made of a material having a desired level of purity appropriate for applications such as drug or bioprocessing that can tolerate a wide temperature range, such as down to liquid nitrogen temperatures. For example, preferably the material of the rigid frame bag is not polyethylene (PE). Each layer of film can be a single layer film or a multiple-layer film. A single layer film can be a film made of one material while a multiple-layer film can include two or more individual layers of the same or different materials.

The frame of the rigid frame bag can have a variety of different shapes, which generally imparts that shape to the shape of the bag. For example, the rigid frame and formed bag may have a rectangular shape with rounded edge, a circular shape, or any other suitable shape. Various shapes are shown in FIGS. 1-6.

In more detail, as shown in FIGS. 1-6 and 10, a periphery of frame 110 can form an enclosed space with one or more openings or apertures exposed to an exterior space. For example, as shown in FIG. 2, the frame 110 includes a first opening 110A and a second opening 110B. The first opening and the second opening form a through hole within the periphery of the frame. A first layer of film (not shown) can be bonded to the frame 110 to cover the first opening 110A, and a second layer of film (also not shown) can be bonded to the opposite side of frame 110 to cover the second opening 110B. The frame, the first layer of film, and the second layer of film form an enclosed space for containing bioprocess fluids. In this way, the frame is disposed between the first layer of film and the second layer of film to form the bag interior, having a volume determined by the sizes of the frame and films. For example, the enclosed space, which is suitable for containing bioprocess fluids, can have a volume ranging from at or about 200 milliliters to at or about 20 liters.

In another embodiment, frame 110 can include one opening. For this embodiment, the structure of the frame can form a recess, and a layer of film can be bonded to the frame to cover the opening. The frame and the layer of film can form an enclosed space for containing bioprocess fluids.

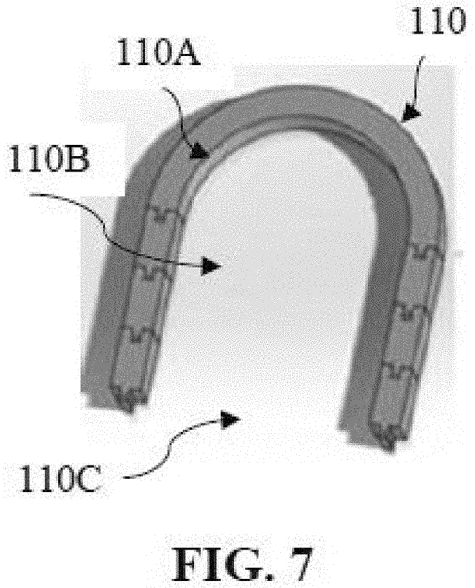

In yet another embodiment as shown in FIG. 7, a periphery of frame 110 can form a partially enclosed space with openings or apertures exposed to an outer space. In this embodiment, frame 110 can include first opening 110A, second opening 110B, and third opening 110C. The first opening and the second opening form a through hole within the periphery of frame 110. A first layer of film (not shown) can be bonded to the frame to cover first opening 110A, and a second layer of film (also not shown) can be bonded to the frame to cover second opening 110B. In addition, a third layer of film (not shown) can also be bonded to frame 110 to cover the third opening 110C. In this way, the frame, the first layer of film, the second layer of film, and the third layer of film form an enclosed space for containing bioprocess fluids.

In various embodiments, the rigid frame bag may include one or more fittings. Each fitting can include one or more valves as may be needed depending on the application. The fittings are configured to fill the rigid frame bag with a bioprocess fluid or to drain the bioprocess fluid from the rigid frame bag. Bioprocess fluids can be directed into or out of the rigid frame bag through the fittings when, for example, a valve of the fitting is open (such as being pressed to open). The fittings and the frame can be made of the same material or compatible materials. In one embodiment, the fittings and the frame can be integrally formed into a unitary piece, and can be formed, for example by injection molding.

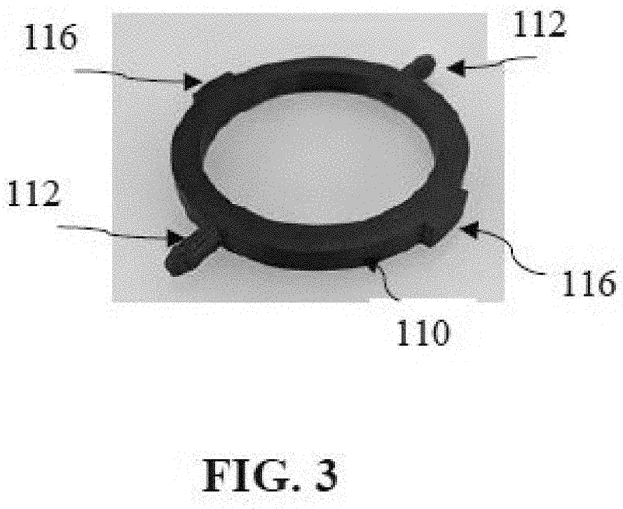

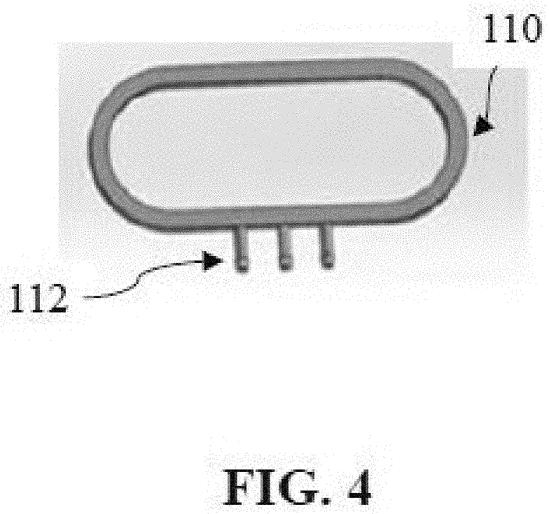

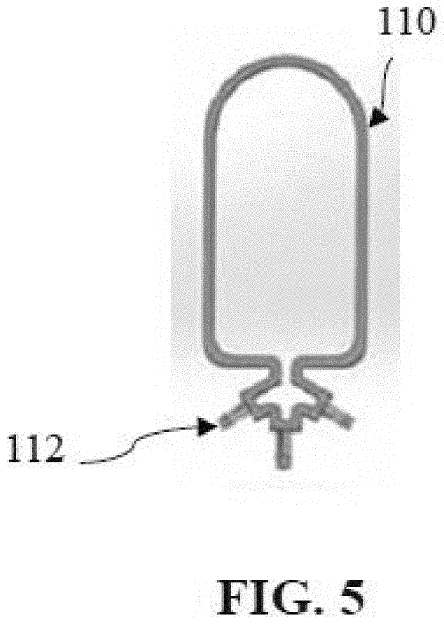

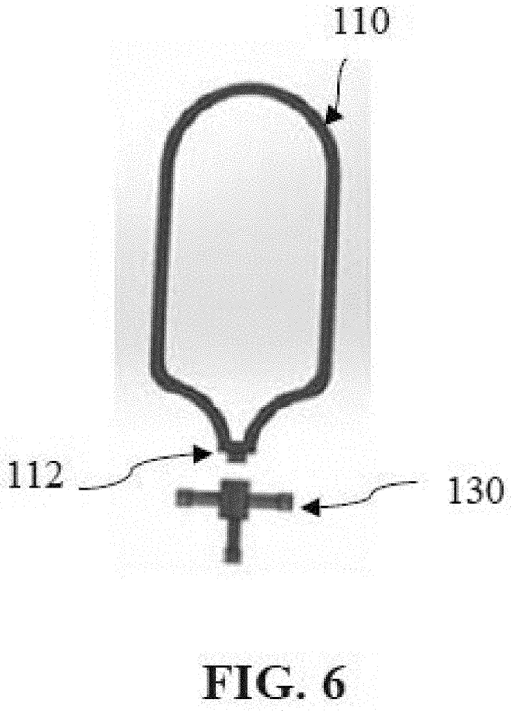

As shown in FIGS. 1-6, rigid frame bag 100 can include one or more fittings 112 along the periphery of frame 110. For example, the rigid frame bag in FIG. 2 and FIG. 3 has 2 fittings, while that shown in FIG. 4 and FIG. 5 has three. Each fitting can be configured to connect to a tube (such as a silicon tube) to fill the rigid frame bag with the bioprocess fluids or drain the bioprocess fluids from the rigid frame bag. Alternatively, as shown in FIG. 6, frame 100 includes one fitting 112 that can be connected to connector 130 having its own fittings, which can then be connected to suitable tubing. In this way, for example, connector 130 can be attached to fitting 112 in order to fill the rigid frame bag and the removed for transportation or storage. When the contents are to be dispensed from the bag, the same or different connector can then be attached to fitting 112.

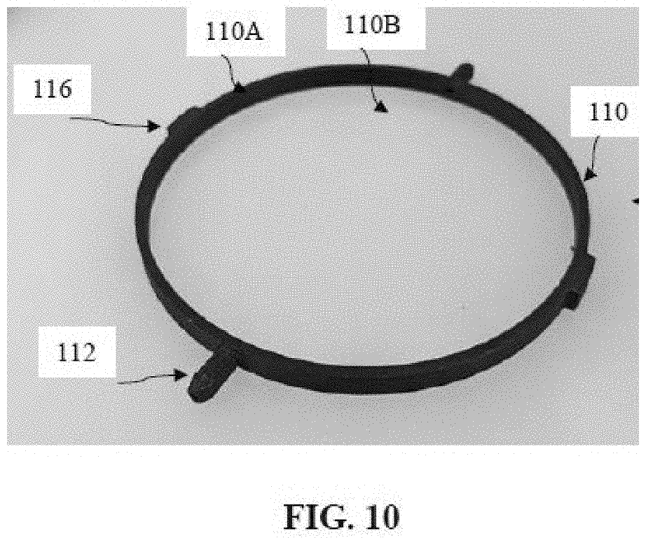

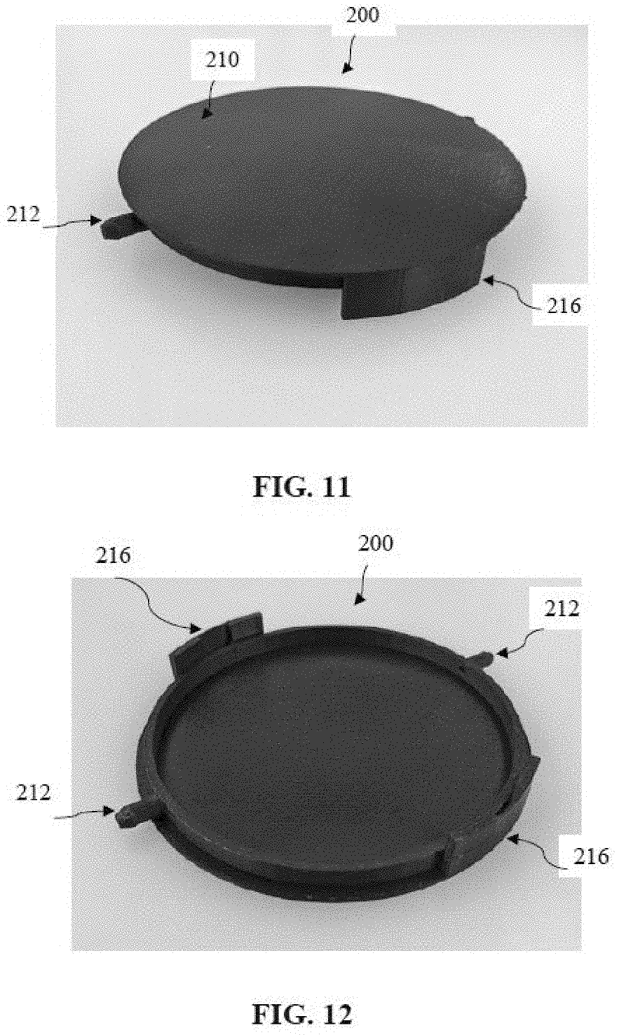

In addition, as shown in FIGS. 3 and 10, frame 110 can include one or more interlocking mechanisms 116 to secure the frame to a cassette of cap. For example, interlocking mechanism(s) 116 shown in FIG. 10 can work with the interlocking mechanisms 216 of cap 200 shown in FIG. 11 to secure the frame to the cap, which is described in more detail below.

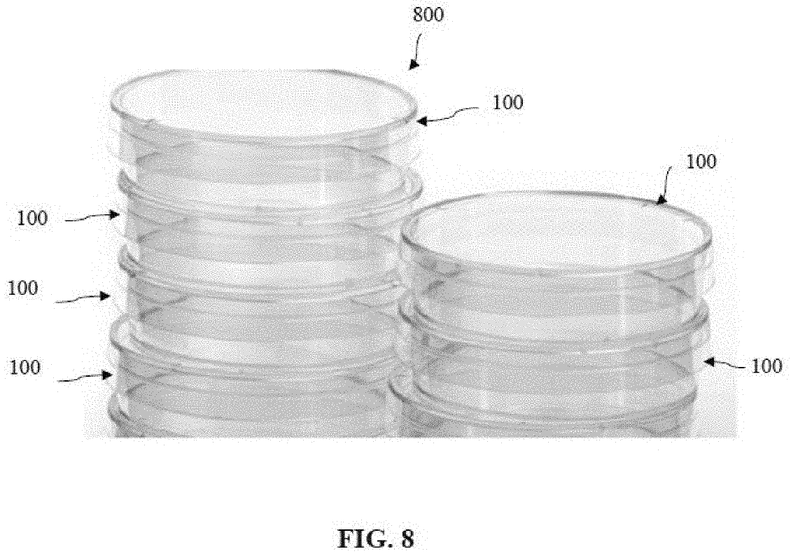

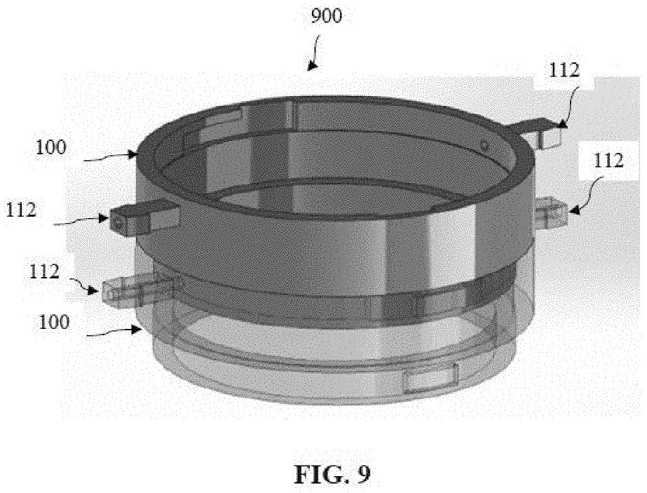

The rigidity of the frame that forms the rigid frame bag enables multiple bags to be stackable. For example, FIG. 8 and FIG. 9 are perspective views of stacks 800 and 900 respectively that include a plurality of individual rigid frame bags 100. By stacking the rigid frame bags in this way, the film layers of the bags can be protected during storage and transportation.

In some embodiments, a cassette or container (such as a cover or cap) can be included to cover, protect, and/or seal off the fittings, such as those discussed above, from the outside environment. The use of a cassette can, for example, keep the fittings of the rigid frame bag clean. FIG. 11 is a top perspective view of cassette 200 while FIG. 12 is a bottom perspective view of the cassette of FIG. 11. As shown, cassette 200 can also include one or more interlocking mechanisms 216. In use, interlocking mechanisms 116 of frame 100 (see FIG. 10) and interlocking mechanisms 216 of cassette 200 are configured to interlock with each other using, for example, a snap-fit connection that can be joined or attached by rotation.

As shown, the cassette is configured to cover the frame or the rigid frame bad, thereby protecting the integrity of the bag. For example, cassette 200 shown in FIG. 11 is configured to cover the first layer of film that covers first opening 110A of frame 110 shown in FIG. 10 or the second layer of film that covers second opening 110B of frame 110. In one embodiment, the cassette can have a flat surface 210, which may facilitate easy shipping and/or storage of the rigid frame bag. For example, the cassette can be made of plastic to protect the rigid frame bag. In another embodiment, the cassette can be made of metal (e.g., stainless steel or the like) to facilitate a freezing and/or thawing process of the contents of the rigid frame bag.

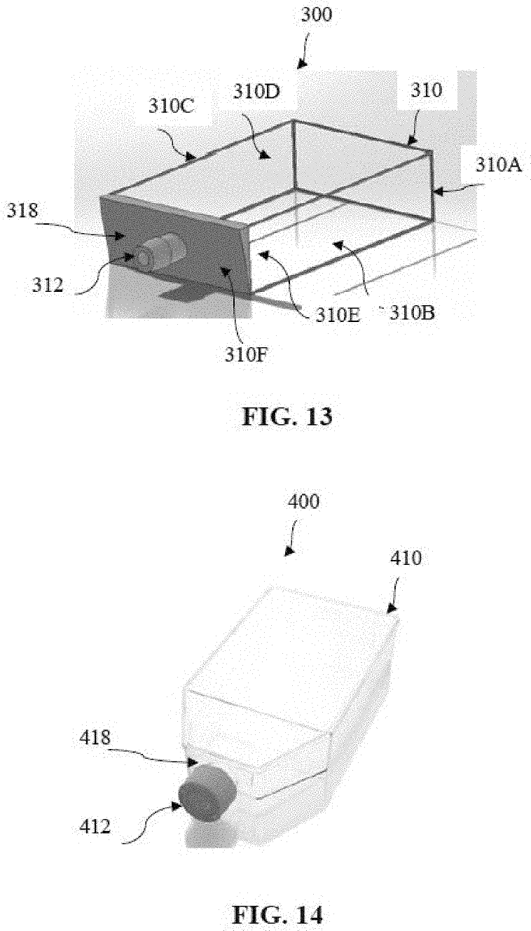

In another embodiment, the rigid frame bag described herein may comprise a frame that includes a plurality of openings. A specific example is shown in FIG. 13 and FIG. 14, which are perspective views of a 3D rigid frame bag 300 and 400 respectively that includes a plurality of openings or facets 310A-310F. One of the openings, such as opening 310F, can be covered by plate 318, which can provide additional rigidity to the 3D structure and can further include fitting 312 disposed in the middle of the plate. It will be appreciated that the rigid frame bag 300 can include more than one fitting and that other openings besides opening 310F can be covered by a plate. As shown, frame 310 includes three or more or any suitable number of openings or facets. For example, frame 310 can have a pyramid shape, a cubic shape, a hexahedron shape, or the like. Except the opening 310F that is covered by the plate 318, layers of film can be bonded to frame 310 to cover openings 310A-310E. The frame, the plate, and the layers of film can form an enclosed space for containing bioprocess fluids.

For this embodiment, plate 318, frame 310, and/or fitting 312 can be made of the same material or compatible materials. As a specific example, plate 318, frame 310, and/or fitting 312 can be integrally formed, such as by using injection molding. Furthermore, rigid frame bag 400 can have structures and/or functionalities similar to those described above, such as rigid frame bag 300, with the exception that rigid frame bag 400 has more openings than and creates a larger 3-dimensional shape.

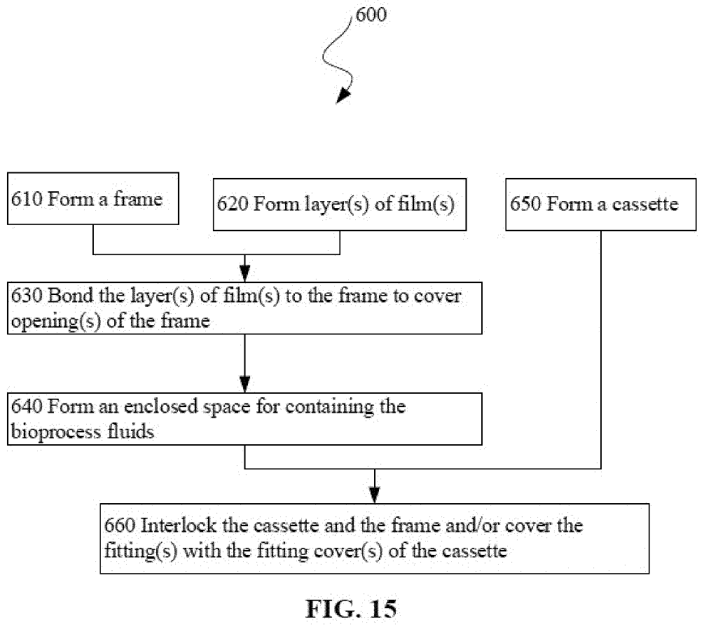

FIG. 15 is a flowchart illustrating a method 600 of forming a bioprocess bag for containing bioprocess fluids. Although illustrated as discrete blocks, various blocks may be divided into additional blocks, combined into fewer blocks, or eliminated, depending on the desired implementation. The bioprocess bag can be any of the rigid frame bags described above, including those shown in FIGS. 1-14.

As shown in the flowchart of FIG. 15, method 600 can begin at 610, 620, or 650. Block 610 can refer to forming a frame having opening(s). Block 620 can refer to forming layer(s) of film(s). Block 650 can refer to forming a cassette, such as cassette 200 of FIGS. 11 and 12.

Blocks 610 and 620 may be followed by Block 630. Block 630 can refer to bonding the layer(s) of film to the frame to cover the opening(s) of the frame. Block 630 may be followed by Block 640.

Block 640 can refer to forming an enclosed space for containing the bioprocess fluids by the frame and the layer(s) of film(s). Blocks 640 and 650 may be followed by Block 660.

Block 660 can refer to interlocking the cassette and the frame using e.g., snap-fit or any other suitable mechanism. Block 660 can also refer to covering the fitting(s) with fitting cover(s) of the cap. Block 660 can further refer to covering one or more sides of the frame with the cassette, and/or covering one or more layer(s) of the film(s) with the cassette. In this way, method 600 can provide bags with expandable volume, flexible connections, better drainage, and/or reduced materials of construction (e.g., plastics or the like).

Aspects:

It is understood that any one of aspects 1-18 can be combined with any one of aspects 19-22 and 23-26. Any one of aspects 19-22 can be combined with any one of aspects 23-26.

Aspect 1. A bioprocess bag for containing bioprocess fluids, comprising: a frame having a first opening and a second opening; a first layer of film bonded to the frame to cover the first opening; and a second layer of film bonded to the frame to cover the second opening, wherein the frame, the first layer of film, and the second layer of film form an enclosed space for containing the bioprocess fluids, the frame is disposed between the first layer of film and the second layer of film.

Aspect 2. The bag of aspect 1, wherein the frame, the first layer of film, and the second layer of film are made of a same material or compatible materials.

Aspect 3. The bag of aspect 1 or aspect 2, wherein the frame, the first layer of film, and the second layer of film are made of fluoropolymer.

Aspect 4. The bag of any one of aspects 1-3, wherein the first layer of film is a single layer film, and the second layer of film is a single layer film.

Aspect 5. The bag of any one of aspects 1-3, wherein the first layer of film is a multiple-layer film.

Aspect 6. The bag of any one of aspects 1-3 and 5, wherein the second layer of film is a multiple-layer film.

Aspect 7. The bag of any one of aspects 1-6, wherein a minimum thickness of the frame is at or about 30 thousandth of an inch.

Aspect 8. The bag of any one of aspects 1-7, wherein a maximum thickness of the frame is at or about three inches.

Aspect 9. The bag of any one of aspects 1-8, wherein a minimum thickness of the first layer of film or the second layer of film is at or about three thousandth of an inch.

Aspect 10. The bag of any one of aspects 1-9, wherein a maximum thickness of the first layer of film or the second layer of film is at or about 25 thousandth of an inch.

Aspect 11. The bag of any one of aspects 1-10, further comprising: at least one fitting, wherein the at least one fitting is configured to fill the bag with the bioprocess fluids or drain the bioprocess fluids from the bag.

Aspect 12. The bag of aspect 11, wherein the at least one fitting and the frame are integrally formed using injection molding.

Aspect 13. The bag of aspect 11 or aspect 12, further comprising: a cap having at least one fitting cover, wherein the at least one fitting cover is configured to protect the at least one fitting.

Aspect 14. The bag of any one of aspects 1-13, wherein the frame includes an interlocking mechanism, the bag further comprises: a cap including an interlocking mechanism, wherein the interlocking mechanism of the frame and the interlocking mechanism of the cover are configured to interlock with each other using snap-fit.

Aspect 15. The bag of aspect 14, wherein the cap is configured to cover the first layer of film that covers the first opening or the second layer of film that covers the second opening of the frame.

Aspect 16. The bag of aspect 14 or aspect 15, wherein the cap has a flat surface.

Aspect 17. The bag of any one of aspects 14-16, wherein the cap is made of plastic.

Aspect 18. The bag of any one of aspects 14-17, wherein the cap is made of stainless steel.

Aspect 19. A bioprocess bag for containing bioprocess fluids, comprising: a frame having a front opening and a plurality of openings; a plate configured to cover the front opening; and a first layer of film, a second layer of film, and a third layer of film bonded to the frame to cover each opening of the plurality of openings, respectively; wherein the plate, the frame, the first layer of film, the second layer of film, and the third layer of film form an enclosed space for containing the bioprocess fluids.

Aspect 20. The bag of aspect 19, further comprising: a fourth layer of film and a fifth layer of film, wherein the first layer of film, the second layer of film, the third layer of film, the fourth layer of film, and the fifth layer of film are bonded to the frame to cover each opening of the plurality of openings, respectively; wherein the frame, the plate, the first layer of film, the second layer of film, the third layer of film, the fourth layer of film, and the fifth layer of film form the enclosed space for containing the bioprocess fluids.

Aspect 21. The bag of aspect 19 or aspect 20, wherein the plate includes at least one fitting, the at least one fitting is configured to fill the bag with the bioprocess fluids or drain the bioprocess fluids from the bag.

Aspect 22. The bag of any one of aspects 19-21, wherein the plate and the frame are integrally formed using injection molding.

Aspect 23. A method of forming a bioprocess bag for containing bioprocess fluids, the method comprising: forming a frame having a first opening and a second opening; forming a first layer of film and a second layer of film; bonding the first layer of film to the frame to cover the first opening; bonding the second layer of film to the frame to cover the second opening; and forming an enclosed space for containing the bioprocess fluids by the frame, the first layer of film, and the second layer of film, wherein the frame is disposed between the first layer of film and the second layer of film.

Aspect 24. The method of aspect 23, further comprising: forming a cap having a flat surface; and interlocking the cap and the frame using snap-fit.

Aspect 25. The method of aspect 24, further comprising: covering at least one fitting with at least one fitting cover of the cap.

Aspect 26. The method of aspect 24 or aspect 25, further comprising: covering the first layer of film that covers the first opening or the second layer of film that covers the second opening of the frame with the cap.

The examples disclosed in this application are to be considered in all respects as illustrative and not limitative. The terminology used in this specification is intended to describe particular embodiments and is not intended to be limiting. The terms “a,” “an,” and “the” include the plural forms as well, unless clearly indicated otherwise. The term “or” includes “and/or” unless clearly indicated otherwise. The terms “comprises” and/or “comprising,” when used in this specification, specify the presence of the stated features, integers, steps, operations, elements, and/or components, but do not preclude the presence or addition of one or more other features, integers, steps, operations, elements, and/or components.

With regard to the preceding description, it is to be understood that changes may be made in detail, especially in matters of the construction materials employed and the shape, size, and arrangement of parts without departing from the scope of the present disclosure. This specification and the embodiments described are exemplary only, with the true scope and spirit of the disclosure being indicated by the claims that follow.

What is claimed is:

1. A bioprocess bag for containing a bioprocess fluid, comprising:
a frame having a first opening and a second opening,
a first layer of film bonded to the frame to cover the first opening, and
a second layer of film bonded to the frame to cover the second opening,
wherein the frame, the first layer of film, and the second layer of film form an enclosed space for containing the bioprocess fluid,
the frame being disposed between the first layer of film and the second layer of film.

2. The bag of claim 1, wherein the frame, the first layer of film, and the second layer of film are made of a same material or compatible materials.

3. The bag of claim 1, wherein the frame, the first layer of film, and the second layer of film are made of fluoropolymer.

4. The bag of claim 1, wherein the first layer of film is a single layer film, and the second layer of film is a single layer film.

5. The bag of claim 1, wherein the first layer of film is a multiple-layer film.

6. The bag of claim 1, wherein the second layer of film is a multiple-layer film.

7. The bag of claim 1, wherein a minimum thickness of the first layer of film or the second layer of film is at or about three thousandth of an inch.

8. The bag of claim 1, wherein a maximum thickness of the first layer of film or the second layer of film is at or about 25 thousandth of an inch.

9. The bag of claim 1, further comprising:
at least one fitting,
wherein the at least one fitting is configured to fill the bag with the bioprocess fluids or drain the bioprocess fluids from the bag.

10. The bag of claim 9, wherein the at least one fitting and the frame are integrally formed using injection molding.

11. The bag of claim 9, further comprising:
a cap having at least one fitting cover,
wherein the at least one fitting cover is configured to protect the at least one fitting.

12. The bag of claim 1, wherein the frame includes an interlocking mechanism, the bag further comprising:
a cap including an interlocking mechanism,
wherein the interlocking mechanism of the frame and the interlocking mechanism of the cover are configured to interlock with each other using snap-fit.

13. The bag of claim 12, wherein the cap is configured to cover the first layer of film that covers the first opening or the second layer of film that covers the second opening of the frame.

14. A bioprocess bag for containing bioprocess fluids, comprising:
a frame having a front opening and a plurality of openings,
a plate configured to cover the front opening, and
a first layer of film, a second layer of film, and a third layer of film bonded to the frame to cover each opening of the plurality of openings,
wherein the plate, the frame, the first layer of film, the second layer of film, and the third layer of film form an enclosed space for containing the bioprocess fluids.

15. The bag of claim 14, further comprising:
a fourth layer of film and a fifth layer of film,
wherein the first layer of film, the second layer of film, the third layer of film, the fourth layer of film, and the fifth layer of film are bonded to the frame to cover each opening of the plurality of openings, and
wherein the frame, the plate, the first layer of film, the second layer of film, the third layer of film, the fourth layer of film, and the fifth layer of film form the enclosed space for containing the bioprocess fluids.

16. The bag of claim 14, wherein the plate includes at least one fitting, the at least one fitting being configured to fill the bag with the bioprocess fluids or drain the bioprocess fluids from the bag.

17. The bag of claim 14, wherein the plate and the frame are integrally formed using injection molding.

18. A method of forming a bioprocess bag for containing bioprocess fluids, the method comprising:
forming a frame having a first opening and a second opening,
forming a first layer of film and a second layer of film,
bonding the first layer of film to the frame to cover the first opening,
bonding the second layer of film to the frame to cover the second opening; and
forming an enclosed space for containing the bioprocess fluids by the frame, the first layer of film, and the second layer of film,
wherein the frame is disposed between the first layer of film and the second layer of film.

19. The method of claim 18, further comprising:
forming a cap having a flat surface; and
interlocking the cap and the frame using snap-fit.

20. The method of claim 18, further comprising:
covering the first layer of film that covers the first opening or the second layer of film that covers the second opening of the frame with the cap.
